(19) United States
(12) Patent Application Publication
(10) Pub. No.: US 2014/0358078 A1
(43) Pub. Date: Dec. 4, 2014

(54) CLOSURE, IN PARTICULAR A SYRINGE CLOSURE FOR CLOSING A DISTAL OPENING OF A SYRINGE BODY IN A SEALING MANNER

(71) Applicant: Schott Schweiz AG, St. Gallen (CH)

(72) Inventors: Bastian Fischer, St. Gallen (CH); Christoph Huber, St. Gallen (CH); Edwin Schellenberg, Neukirch-Egnach (CH); Heinz Häberlein, Happerswil (CH); Mustafa Kücük, Staad (CH); Thomas Fässler, Romanshorn (CH)

(73) Assignee: SCHOTT SCHWEIZ AG, St. Gallen (CH)

(21) Appl. No.: 14/299,684

(22) Filed: Jun. 9, 2014

Related U.S. Application Data
(63) Continuation of application No. PCT/EP2012/005063, filed on Dec. 7, 2012.

(30) Foreign Application Priority Data
Dec. 9, 2011 (DE) 10 2011 120 637.3

(51) Int. Cl. A61M 5/31; A61M 5/50

(52) U.S. Cl. CPC A61M 5/3134; A61M 5/50; A61M 2005/3104; A61M 5/347
USPC 604/111

(57) ABSTRACT

A closure for closing in a sealing manner a distal opening on a syringe body is provided. The closure includes a fastening element that is or can be arranged on the syringe body around the distal opening and a closure cap that can be loosely connected to the fastening element and closing in a sealing manner the distal opening. The fastening element includes an inner thread that intermeshes with an outer thread on the closure cap, resulting in an initial state wherein the outer thread is formed on at least part of the closure cap. The part is under tension in such a way that after the closure cap is unscrewed from the inner thread the part under tension is pressed out over the inner thread of the fastening element such that the closure cap cannot be returned to the initial state once the closure cap is unscrewed.

### CROSS REFERENCE TO RELATED APPLICATIONS

This is a continuation of PCT Application No. PCT/EP2012/005063, entitled “CLOSURE, PARTICULARLY A SYRINGE CAP FOR CLOSING A DISTAL OPENING OF A SYRINGE BODY IN A SEALING MANNER”, filed Dec. 7, 2012, which is incorporated herein by reference.

### BACKGROUND OF THE INVENTION

1. Field of the Invention

The present invention relates to a closure, in particular a syringe closure for closing in a sealing manner a distal opening, such as a syringe cone, on a syringe body.

2. Description of the Related Art

Syringe closures are known from a multitude of patent specifications. U.S. Pat. No. 6,190,364, for example, describes a syringe closure for a syringe body. The syringe body itself comprises a distal syringe and a distal opening extending through same, in particular in the embodiment of a syringe cone so that a liquid which is located inside the syringe body can leave the syringe body through the distal opening, or respectively the syringe cone. The syringe body moreover comprises a fastening element which is arranged or can be arranged on the syringe body. This fastening element can be part of a single component syringe or may be placed on the distal tip of the syringe body and be firmly connected with same. The fastening element moreover has threads or bayonet structures which interact with the threads of a two-component closure cap in order to hold the closure cap on the fastening element. The closure cap surrounds the region of the distal syringe, or respectively the syringe cone, and closes and seals the distal opening of the syringe cone. To be able to inject the syringe liquid the closure cap in U.S. Pat. No. 6,190,364 is unscrewed from the fastening element and an injection cannula is secured on the syringe body in such a manner that a needle opening extending through the injection cannula forms a flow connection with the distal opening of the injection body.

One problem with syringe closure elements of this type is that it must be ensured that they cannot be reused in certain fields of applications, for example with another medication or for another application. Such second use would present a high risk of infection of the patient due to contamination, for example though foreign particles and germs. An additional risk exists in that when reusing the syringe closure on a syringe body other than the original one, a contamination of the second medication occurs since traces of the original medication may be present in the closure cap which could be transferred into the second medication and could lead to incorrect treatment of the patient.

To eliminate possible manipulations and a second use of the syringe closure according to U.S. Pat. No. 6,190,364, the syringe closure is provided with a sealing strip which is detachably connected with the fastening element as well as with the closure cap so that the sealing strip tears when the closure cap is released from the fastening element. With a closure according to U.S. Pat. No. 6,190,364 it is disadvantageous that several separate parts must be manufactured which are subsequently connected with each other, thereby incurring additional costs.

A syringe closure for sealing and closing has therefore become known from EP 1 600 190 A1, which on the one hand recognizes manipulations at the syringe opening, and whereby on the other hand manufacture has been significantly simplified compared to U.S. Pat. No. 6,190,364. For this purpose the fastening device in EP 1 600 190 A1 was provided with a locking device which locks with a locking device of the closure cap. Moreover the locking device of the fastening element as well as that of the closure cap are designed such that they cannot be unlocked without damaging them. This means that after unlocking, a damage free relocking is not possible. Even though damage-free relocking is ruled out in EP 1 600 190 A1, it is possible to again lock the closure cap with the fastening element by accepting damage. Protection from multi-use—in order to ensure single use for a syringe—is also not provided by EP 1 600 190 A1.

A closure for a pre-fillable, single use plastic syringe having a syringe body and a syringe needle which is firmly connected with the syringe body's delivery end, consisting of a rigid outer plastic protective cap for the needle, formed of a hard elastic material which can be placed directly onto the syringe body, surrounding the syringe needle and which, on the inside at least in the region of the needle tip, is provided with a sealing lining consisting of a soft elastic material has become known from EP 1 101 505, wherein the plastic protective cap for the needle is welded or glued directly circumferentially with the syringe body, whereby an intended separation point is provided in the welded or glued seam.

What is needed in the art is a closure configured to avoid the disadvantages according to the current state of the art and to specify a closure element to seal, in particular a syringe opening which functions as a sterility barrier as well as protection from multiple uses of the closure cap. Moreover, these functions are to be provided with the fewest possible components and with the associated low manufacturing costs.

### SUMMARY OF THE INVENTION

The present invention provides a closure, for example a syringe closure, for sealing a distal opening, for example of a syringe cone of a syringe body. The syringe closure includes a fastening element which is arranged or can be arranged on the syringe body around the distal opening, for example around the syringe cone and a closure cap which closes the distal opening, for example the syringe cone, in a sealing manner and which is connected detachably with the fastening element. According to the present invention, the fastening element includes an inside thread which engages with an outside thread of the closure cap, resulting in an initial state. The outer thread is formed from at least one part of the closure cap, and the part is under tension in such a way that after unscrewing the closure cap from the fastening element the part under pressure is pressed out over the inside thread such that the closure cap cannot again be returned to the initial state once the closure cap is unscrewed. The pre-tension is introduced through the assembly process. The hard component is already produced in the injection molding or manufacturing process in an expanded state and is pre-tensioned by being brought together with the inside thread of the fastening element.

In the case of the plastic syringes, the fastening element or the fastening ring or respectively the Luer collar is sprayed onto the syringe body so that a single component results. In the case of glass syringes the fastening element, or respectively the fastening ring, is a separate component which, for example, locks in place on a bead on the glass syringe at a predetermined or a defined height. By the tightness of the connection of the closure cap and the distal opening, or respectively the cone of the glass syringe, the predefined distance and engagement is also ensured.

Due to the fact that the threaded components of the closure cap are pushed outward after being unscrewed according to the present invention, repeated use after removal of the closure cap is no longer possible.

The inventive syringe closure can be used for a syringe body formed of a glass material, but also for one formed of a thermoplastic material.

As described previously, the fastening element on a glass syringe body is a separate component, for example a fastening ring, which locks into a bead on the glass material at a predefined height, whereas on a plastic syringe body, in this case a thermoplastic syringe body, the fasting element is part of the syringe body.

In one embodiment of the present invention, the closure cap includes an elastomer base element with which the distal opening, for example the syringe cone, is closed, as well as a region consisting of a hard component, a so-called hard region. Pushing out the part of the closure cap over the inside thread occurs, for example in the region which consists of the hard component. The tension can hereby be introduced into the hard component itself. In one alternative embodiment, the function of pushing out of the hard component during unscrewing is achieved through specially formed elastomer components, for example the base material or respectively the base element or base component.

The hard component of the closure cap which is provided with the outside thread can be connected mechanically with the base component, which can consists of an elastomer, in particular in a region surrounding the distal opening, for example of the syringe body. The mechanical connection of the threaded component or the hard component with the base component offers the advantage that over-tightening of the base component is prevented. In order to develop the closure cap to be capable of being sterilized in all conventional sterilization methods, for example gamma radiation, ethylene oxide sterilization (ETO) and steam at temperatures of 121° C. and higher, as well as electron radiation, the elastomers of the base material are advantageously selected accordingly. This allows broad use, in addition to glass syringes, also of thermoplastic syringes.

The handling region of the closure cap is, for example, integrated into the base material or the base component of the closure cap. The hard region which is provided with the outside thread is, for example, configured as an expansion element which expands after being unscrewed from the closure cap. The expanded expansion element then has a greater diameter than the inside diameter of the inside thread. This prevents the closure cap from being again screwed onto the syringe cone with a distal opening.

To guarantee protection from multiple uses a further developed embodiment of the present invention provides that the base component only surrounds a small part of the syringe cone with the distal opening. This ensures that the base component alone, without the threaded component, cannot hold itself on the cone. The threaded component of the outside thread, for example, includes only one single cam so that the closure cap only hooks into the inside thread of the syringe and can be turned off merely by a quarter turn. In order to prevent slipping or detachment from the inside thread, for example during storage or transport, it may be provided that the threaded component in an embodiment of this type of the closure cap is connected with the inside thread through a shaping process.

All materials used for the closure cap, that is the elastomer as well as also the hard components, are selected so that they can be used for a pharmaceutical application. The materials, for example, have a USP-class-VI-classification. Moreover they can also be colored.

As mentioned previously elastomers which are permissible for pharmaceutical applications and butyl rubber can be used. As hard materials, in particular for the hard component, all permissible thermoplastics can be utilized. For example, thermoplastics which can survive as many types of sterilization as possible without undergoing significant geometric or material changes are advantageous. Exemplary thermoplastics are polycarbonate, polyetheretherketone, polyetherimide, polyether block amide, and copolyester.

In addition to the device according to the present invention, a method for the production of the syringe closure is also specified. For the hard component a conventional injection molding process can be used. For the elastomer component, various methods may be used, such as the compression or vulcanization method. The elastomer component can moreover also be produced in a transfer-injection or injection molding process. The two components are combined and connected with each other in an additional process step using an assembly system.

The sealing cap for sealing of the distal opening, for example the syringe closure, includes, according to a second aspect of the present invention, a hard region and a region which is formed by an elastomer, in other words an elastomer component, wherein the connection between the elastomer component and the hard component can be positive and can be achieved in various ways.

The various possibilities of a positive connection are discussed in detail below. In one arrangement of the closure cap the hard component, which is for example in the embodiment of a bushing, such as an outer cap, is provided which includes a through-opening in an axial direction. The opening can be oval, circular or polygonal.

In the first arrangement, the hard region or respectively the hard component, for example the bushing, such as the outer cap, includes at least one axial section which includes at least one recess, for example at least one opening and/or groove distributed over a circumference of the section.

The distal region of the outside geometry of the elastomer component is arranged such that it is congruent with the continuous opening of the hard component. In contrast to the hard component, the distal section of the elastomer component, which is also referred to as the base component, includes one or several radially projecting protrusions or longitudinally shaped ribs, which in number correspond to the longitudinally shaped recesses, for example openings inside the hard component. In an assembled or fitted condition the protrusion or respectively ribs of the elastomer components are then accommodated by the openings and/or grooves of the hard components.

The outside diameter of the ribs or protrusions is advantageously dimensioned so that it is at least as large as the outside diameter of the hard component, preferably a little larger. Due to the projection of the ribs or protrusions over the hard components an anti-slip surface is created which considerably improves gripping and turning of the closure cap. Additional components for this purpose may furthermore be foregone. Due to the engagement of the ribs or protrusions into the windows both components are interlocked axially and radially with each other. By unscrewing the closure cap from the fastening element the elastomer component is forcibly moved together with the hard component in the axial and the radial direction and is therefore separated from the syringe cone.

In an additional arrangement of a closure cap, the hard component includes an opening, such as a continuous stepped opening in the axial direction, wherein the opening of the hard component is smaller on the distal side than the opening of the hard component on the proximal side. According to the present invention the opening on the proximal side is ring-shaped and the opening in at least one axial section is not circular in its cross section, for example oval or polygonal.

In one arrangement with two axial sections of the opening, the opening in the distal region with the non-circular cross section, or respectively a cross sectional area with a non-circular inside contour, includes ribs or protrusions which are directed inwards, in other words extending radially from the inside contour into the axial opening of the hard component. The number of ribs or protrusions corresponds, for example with the polygonal arrangement of the opening. In such an arrangement the distal region of the outside geometry of the elastomer component or the base component is arranged such that it is congruent with the continuous stepped opening of the hard component, meaning that one section of the elastomer component is not circular in its cross section, for example polygonal. In one embodiment the section with the non-circular outside contour includes grooves into which the ribs or protrusions of the hard region engage in an assembled state. Due to the engagement of the ribs into the grooves, both components are axially and radially interlocked with each other. By unscrewing the closure cap from the fastening element the elastomer component is forcibly moved together with the hard component in the axial and radial direction and is therefore separated from the syringe cone.

In yet another arrangement of a connection of the hard component and the elastomer component, the hard component includes an opening, for example a continuous stepped opening in the axial direction, wherein the openings at the ends of the hard component in the axial direction are larger than that of an opening located between them. In other words the cross section of the intermediate opening in the axial direction has a first cross section (Q₁) which is always smaller than the second cross section (Q₂) and the third cross section (Q₃) of the end regions of the opening in the axial direction. The inside contour of the opening at the ends can be circular, oval or polygonal. The inside contour of the opening in the first axial region is not circular or round, for example polygonal or multi-edged or oval. The elastomer component, or respectively the base component, is configured so that it is congruent with the continuous stepped opening of the hard component, at least in the distal region. This type of arrangement provides an axial interlocking of the hard and the elastomer component or respectively base component.

By unscrewing the closure cap from the fastening element the elastomer component is forcibly moved together with the hard component in the axial direction and is therefore separated from the syringe cone. Due to the non-circular, for example polygonal, arrangement of the two components, rotational security is achieved according to the present invention. This means, if the hard component is turned to be released, the elastomer component or base part turns with it. Moreover, due to the polygonal shape the torque, when unscrewing the closure cap, is reduced since the elastomer component turns along with it. The expenditure of force can thus be greatly reduced.

The technical advantages of all three previously described arrangements are found in an efficient and robust embodiment. The axial and radial connection of both components can be realized in a simple manner. This in turn results in that both components can be produced cost effectively and that the expenditure for the production can be kept low.

According to a fourth aspect of the present invention a closure is to be specified wherein dropping of the closure cap off the syringe opening is avoided. According to the present invention this fourth aspect provided by a closure, in particular a syringe closure for closing in a sealing manner a distal opening, for example of a syringe cone on a syringe body, wherein the closure, in particular the syringe closure includes at least one a fastening element, for example an expansion component having at least one expansion element which is arranged or can be arranged on the syringe body around the distal opening, particularly around the syringe cone, and a closure cap which closes the distal opening, in particular the syringe cone in a sealing manner and which is connected detachably with the fastening element. The closure cap in the region of the fastening element is connected, for example exclusively material-to material-connected with the fastening element, in particular the expansion component, preferably the expansion element, for example welded. This avoids an unintended dropping or coming off of the closure cap. The closure cap can be laser welded and/or ultrasonically welded to the fastening element. The closure cap is material-to material connected with the fastening element, for example in the region of the outside thread or cams of the fastening element.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIGS. 1A-1B illustrate a first embodiment of a syringe and an inventive closure in the initial state, that is closed and sealed, wherein this syringe is a plastic syringe;

FIGS. 2A-2B illustrate a syringe closure in accordance with the first embodiment of FIGS. 1A-1B in the unscrewed open state, wherein the syringe is a plastic syringe;

FIGS. 3A-3D illustrate a base component of a syringe closure according to FIGS. 1A-2B, FIG. 3B is a section view of a base component taken along line I-I in FIG. 3D;

FIGS. 4A-4D illustrate a threaded component of a syringe closure according to FIGS. 1A-2B, FIG. 4B is a section view of a threaded component taken along line I-I in FIG. 4D;

FIGS. 5A-5D illustrate the closure cap according to FIGS. 1A-1B, consisting of base component and hard component with thread, FIG. 5B is a section view of the closure in FIG. 5D taken along line I-I;

FIG. 6 illustrates individual components of the syringe closure according to the first embodiment of the present invention shown in FIGS. 1A-1B, utilized on a syringe which is in the embodiment of a glass syringe;

FIGS. 7A-7D illustrate the base component or base element of the closure cap of a second embodiment of a syringe closure according to the present invention, FIG. 7B is a section view of the base component in FIG. 7D taken along line I-I;

FIGS. 8A-8D illustrate the hard component with a threaded section of a second embodiment of a syringe closure, illustrated in FIGS. 7A-7D according to the present invention;

FIGS. 9A-9D illustrate a closure cap with the components according to FIGS. 7A-8D, FIG. 9B is a section view of the closure cap in FIG. 9D taken along line I-I;

FIG. 10 illustrates a syringe closure according to the second embodiment with components according to FIGS. 7A-9D, utilized on a syringe which is in the embodiment of a glass syringe;

FIGS. 11A-11B illustrate a syringe and inventive closure according to the second embodiment according to FIGS. 7A-10 in the initial state, that is closed and sealed, wherein the syringe is a plastic syringe;

FIGS. 12A-12B illustrate a syringe closure according to the second embodiment in the unscrewed, open state, wherein the syringe is a plastic syringe;

FIGS. 13A1-13C4 illustrate a first embodiment of a closure cap, wherein the hard component includes at least one axial section which has at least one opening and/or groove distributed over a circumference of the section and one thread;

FIGS. 14A1-14C4 illustrate an embodiment as in FIGS. 13A1-13C4, wherein the hard part or component features cams;

FIGS. 15A1-15C4 illustrate a second embodiment of a closure cap, wherein the hard part, or respectively component, includes a thread and an opening extending in an axial direction, in particular a through-opening which has an inside contour in at least one section which is not circular, as well as three axial sections;

FIGS. 16A1-16C4 illustrate an embodiment as in FIGS. 15A1-15C4, wherein the part or component features cams;

FIGS. 17A1-17C4 illustrate a third embodiment of a closure cap, wherein the hard part or component features a thread as well as an opening, in particular a through-opening, extending in the axial direction, which in at least one section features an inside contour which is not circular, as well as an axial section with radial protrusions; and

FIGS. 18A1-18C4 illustrate an embodiment as in FIGS. 17A1-17C4, wherein the hard part or component features cams.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

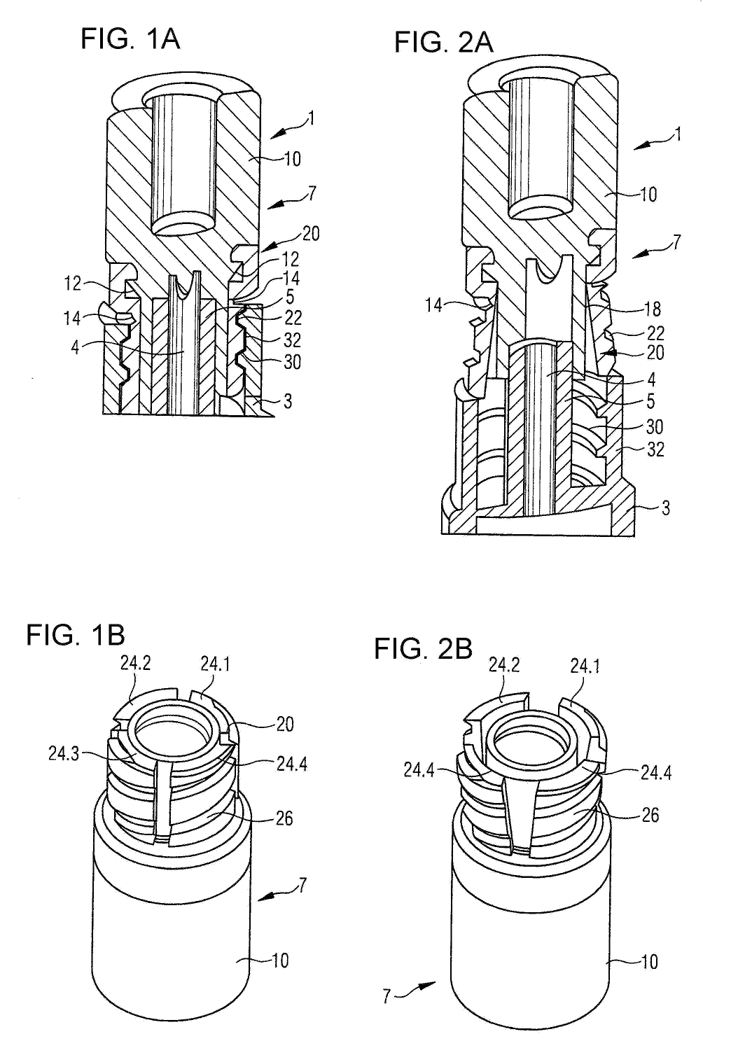

Referring now to the drawings, and more particularly to FIG. 1A, there is shown a complete syringe closure 1 according to a first embodiment of the present invention. Complete syringe closure 1 includes a syringe body 3 with a distal opening 4. Distal opening 4 is surrounded by an elastomer, in particular an elastomer which is permissible in pharmaceutical applications. Distal opening 4 of syringe cone 5 is hereby sealed in a closed state. The elastomer can also be colored. Permissible elastomers are thermoplastic elastomers and butyl rubbers. The elastomers are selected such that the entire closure cap 7 is capable of being sterilized in all conventional sterilization methods, for example gamma radiation, ETO or steam at temperatures of 121° C. and higher, as well as electron radiation. This allows broad use, in addition to glass syringes, also of thermoplastic syringes, as illustrated in FIGS. 1A-2B. In the case of plastic syringes the fastening element 32, or respectively the fastening ring 32, and the plastic syringe or syringe body 3 is a single component as shown.

In addition to the base element or base component 10 the closure cap 7 includes at least one hard part or component 20 as a second component. Hard component 20—as illustrated in detail in 1B—is provided with an outside thread which in the illustrated initial state in FIGS. 1A-1B, that is in the sealed state, engages with inside thread 30 of fastening element 32 of syringe body 3.

The hard component 20 is connected mechanically with the base element or respectively base component 10. For this purpose hard component 20 is provided with a groove which engages with a bead 12 on the base component. The mechanical connection of the two components of closure cap 7 provides that over rotation of the base component 10 is prevented.

As shown in FIG. 1B, the hard component or hard region 20 of closure cap 7 is divided into a total of four expanding elements. In the embodiment illustrated in FIGS. 1A to 2B the hard component or the expansion component or respectively the expansion elements themselves are under tension in the region of notch 14. If closure cap 7 is unscrewed during opening, then the hard component or respectively the expanding element is pressed out over the inside thread, as shown in FIGS. 2A to 2B.

In FIG. 1B, the same reference numbers are used for same components as in FIG. 1A. FIG. 1B merely illustrates closure cap 7, including base component 10 as well as a total of hard regions 20 which in this instance are in the embodiment of expansion elements 24.1, 24.2, 24.3, 24.4 and an outside thread 26 which when screwed down as shown in FIG. 1B engages with inside thread 30 of syringe body 3.

Referring now to FIGS. 2A to 2B, there is shown how the syringe closure in accordance with the first embodiment for a plastic syringe reacts after opening of the closure cap. The same components are again identified with the same reference numbers as in FIGS. 1A and 1B. By unscrewing closure cap 7 with base element 10 and the hard element 20 or respectively the region with outside thread 22 from inside thread 30 of fastening element 32 of syringe body 3, the hard component 20 or respectively hard element which in this instance is under tension in region 14 is pressed out over the inside diameter of fastening element 32 of syringe body 3. It is then no longer possible to screw it in. Clearly illustrated is distal opening 4 of syringe cone 5 of syringe body 3, fastening element 32 of the syringe body which is equipped with inside thread 30, as well as region 18 of base element 10, which as an elastomer fits against the distal opening 4 and seals it tightly in FIG. 1A.

FIG. 2B shows again the sealing element or respectively closure cap 7 consisting of base element 10 and the four expansion elements 24.1, 24.2, 24.3, 24.4 with outside thread 26. In an unscrewed state, the expansion elements expand due to the tension and provide a greater diameter than the inside diameter of fastening element 32 of syringe body 3 in the region of thread 30.

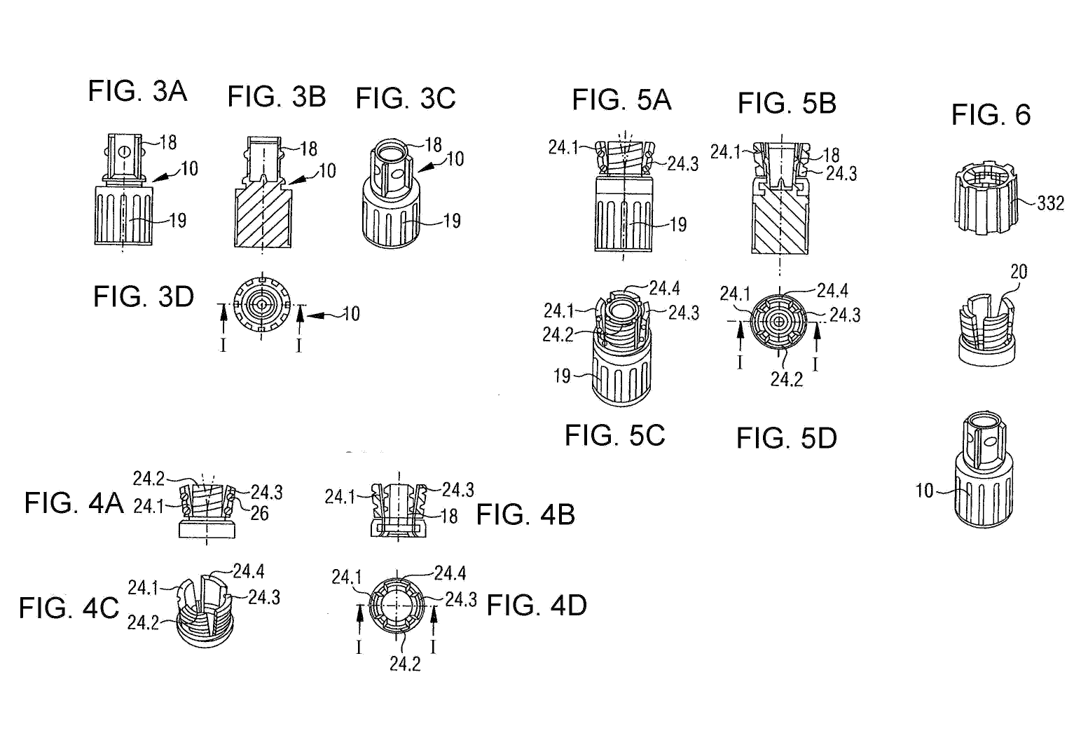

Referring now to FIGS. 3A to 6, there are shown sectional drawings of individual components of the first embodiment of the closure cap with expansion elements, as shown in FIGS. 1A to 2B.

Whereas the closure element or respectively the closure cap (FIGS. 5A-5D) consisting of base element 10 (FIGS. 3A-3D) and hard component (FIGS. 4A-4D) is structurally identical to the embodiment in FIGS. 1A-2B, FIG. 6 illustrates the embodiment for fastening the closure cap on a glass syringe with a fastening ring or respectively fastening element 332 which is separate from the syringe body and which engages on a bead on the glass syringe (not shown).

FIGS. 3A to 3D show base element 10 in a side view (FIG. 3A), sectionally (FIG. 3B) in a three-dimensional depiction (FIG. 3C) as well as in a top view (FIG. 3D). Clearly visible is region 18 which surrounds the distal opening of the syringe cone of the syringe body (not shown). Moreover, as can be seen in FIGS. 3A and 3C, the base element has a gripping region 19 on its outside.

The threaded ring illustrated in FIGS. 4A to 4D, which is also referred to as the hard component 20, is mechanically connected with base element 10. As illustrated in FIGS. 1A to 2B the threaded ring includes a total of four hard regions which are in the embodiment of expansion elements 24.1, 24.2, 24.3, 24.4 which are under tension in region 18 so that they expand when the threaded ring and outside thread 26 are unscrewed from the syringe body. This is clearly visible in FIGS. 4A to 4D.

Referring now to FIGS. 5A to 5D, there is shown the mechanical assembly of base component 10 according to FIGS. 3A to 3D with the threaded ring according to FIGS. 4A to 4D resulting in the closure cap, as illustrated in FIGS. 5A to 5D. Same components as in FIGS. 3A to 4D are used in FIGS. 5A to 5D.

Referring now to FIG. 6, there are shown the individual components of the syringe closure according to the present invention, namely base component 10, and hard component 20 with expansion elements 24.1, 24.2, 24.3, 24.4. In the current example, fastening element 332 with inside thread 30 is a separate component to the syringe body. This embodiment is used primarily with glass syringes where the fastening ring or fastening component 332 is slid over a bead on the glass syringe.

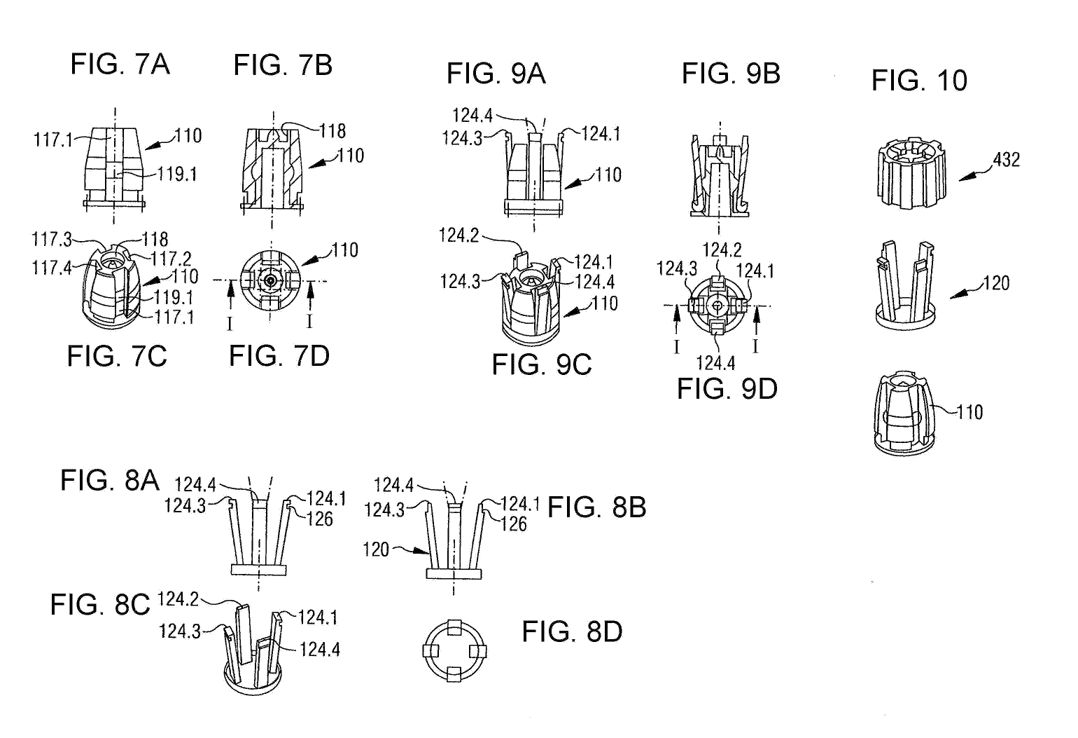

A second embodiment of the present invention is illustrated in FIGS. 7A to 12B. In contrast to the embodiment according to FIGS. 1A to 6 the hard component is longer than base component 110 itself in the embodiment which is shown in FIGS. 7A to 12B so that base component 110 only surrounds a small part of the syringe cone or respectively distal opening 105, so that base component 110 according to FIGS. 7A to 7D cannot be held on the syringe cone with the distal opening without the hard component which is equipped with the thread and which is shown in FIGS. 8A to 8D. The region which surrounds the syringe cone is pictured in FIG. 7B and is identified with 118. It is clearly shorter in length than section 18 in FIGS. 1A to 2D.

The same reference numbers increased by 100 are generally used for same functional components in FIGS. 7A to 12 as in FIGS. 1 to 6. Clearly visible in FIG. 7C are recesses 117.1, 117.2, 117.3, 117.4 in the base body or respectively base element 110 which accommodate the expansion elements of the threaded element, as shown in FIGS. 8A to 8D. Base element 110 moreover includes a bead 119.1 in the region of recesses 117.1, 117.2, 117.3, 117.4 which ensures that, when the closure cap is unscrewed from the distal opening, or respectively the cone opening, the expansion elements are pushed out over the inside diameter of the syringe body. In the embodiment according to FIGS. 7A to 7D as well as FIGS. 8A to 8D the tension is not introduced into the hard element itself, but is provided through the elastomer of the base component. Various views of the hard region, or respectively regions, are shown in FIGS. 8A to 8D. The hard element of the closure cap shown in FIGS. 8A to 8D includes a total of four hard regions in the form of expansion elements 124.1, 124.2, 124.3, 124.4. The hard regions are again provided with an outside thread. However, in the current example this is a single cam 126 in contrast to the several threads of the outside thread according to FIGS. 4A to 4D. The use of only one cam for the outside thread has the advantage that the closure cap with the assistance of the threaded part consisting of only one cam can merely be hooked into inside thread 130 of fastening ring 432 of syringe 103 and can be turned down with only one quarter turn. This guarantees protection from multiple uses since the base component cannot be held on the syringe cone with the distal opening without the threaded part. The embodiment according to FIGS. 7A-10 is again an embodiment having a separate fastening element 432, for example for a glass syringe. In order to prevent slipping or detachment into the initial state—that is into the closed state of the closure cap according to FIGS. 7A to 12—of the syringe body, provision can be made to connect the threaded part of the closure cap through a shaping process with the inside thread of the syringe cone.

The closure element, or respectively the closure cap, which is composed of the components shown in FIGS. 7A to 8D is depicted in FIGS. 9A to 9D. Same components are again identified with same reference numbers as in FIGS. 7A to 8D.

Referring now to FIG. 10, there is shown the assembly of the syringe closure according to the present invention, consisting of base element 110, hard element 120 and fastening element 432 as separate components for a glass syringe.

Similar to FIGS. 1A to 2B, FIGS. 11A to 12B illustrate in a three-dimensional depiction of an embodiment having, on the one hand, the closure in the initial state as shown in FIGS. 1A and 1B, as well as the syringe closure in the open state after unscrewing by a quarter turn of the only one thread of the inventive closure cap according to FIGS. 2A and 2B. This is again a plastic syringe wherein the fastening element 132 is part of syringe body 103.

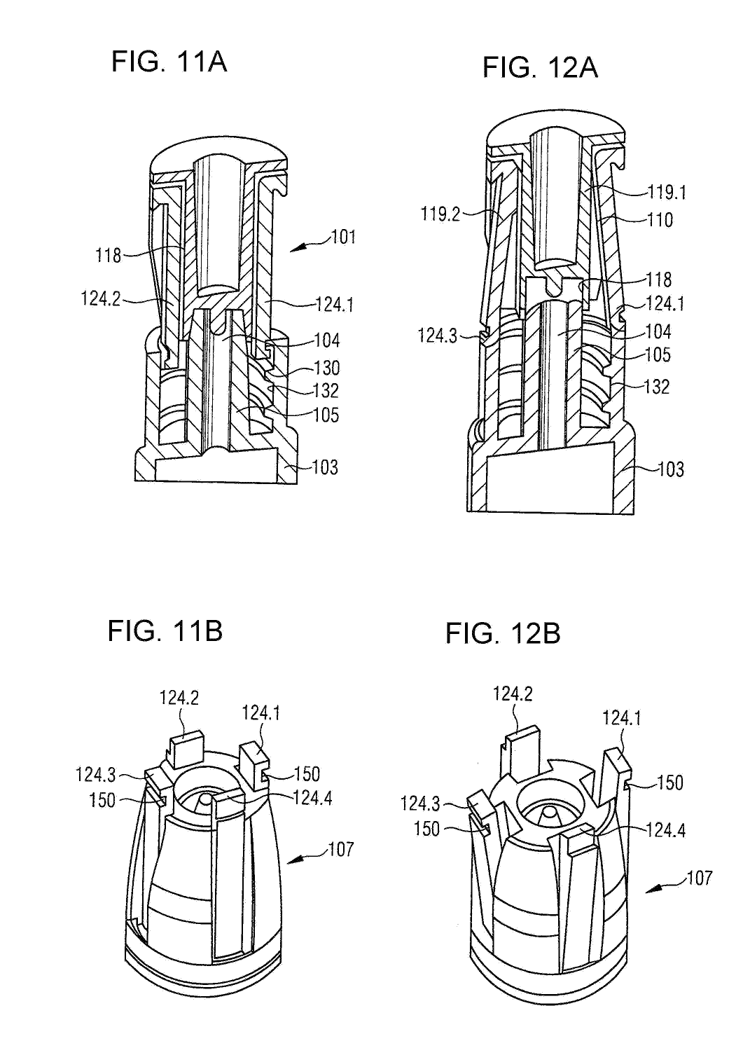

Same components as in FIGS. 7A to 10 are identified with the same reference numbers in FIGS. 11A-11B. Same components as in FIGS. 1A to 6 are identified with reference numbers increased by 100. FIG. 11A in particular illustrates syringe body 103 which is connected with closure cap 107 in the region of expansion elements 124.1, 124.2, 124.3, 124.4. In order to avoid closure cap 107 coming off, closure cap 107 is welded, for example laser welded, in the region of outside thread 122 of expansion elements 124.1, 124.2 with inside thread 130 in the region of fastening element 132 of the syringe body. Two, 124.1, 124.2 of the total of four expansion elements 124.1, 124.2, 124.3, 124.4 are equipped with cams 150 which are illustrated in FIGS. 12B and 11B which can be screwed by a very small amount into inside thread 130 of the syringe body. After just one small turn movement of, for example a quarter turn, the laser weld between the closure cap, the outside thread or respectively cams 150 and inside thread 130 of fastening element 132 breaks open during unscrewing according to FIG. 12A and closure cap 107 drops off the syringe with a distal opening 105 or respectively a syringe opening.

The previously illustrated closure caps 7, 107 are closure caps wherein according to FIGS. 3A to 6 the elastic element is anchored inside a groove in the hard component preventing axial displacement. This embodiment moreover features a protrusion of the elastic element, which provides an excellent grip and supports unscrewing of the closure cap.

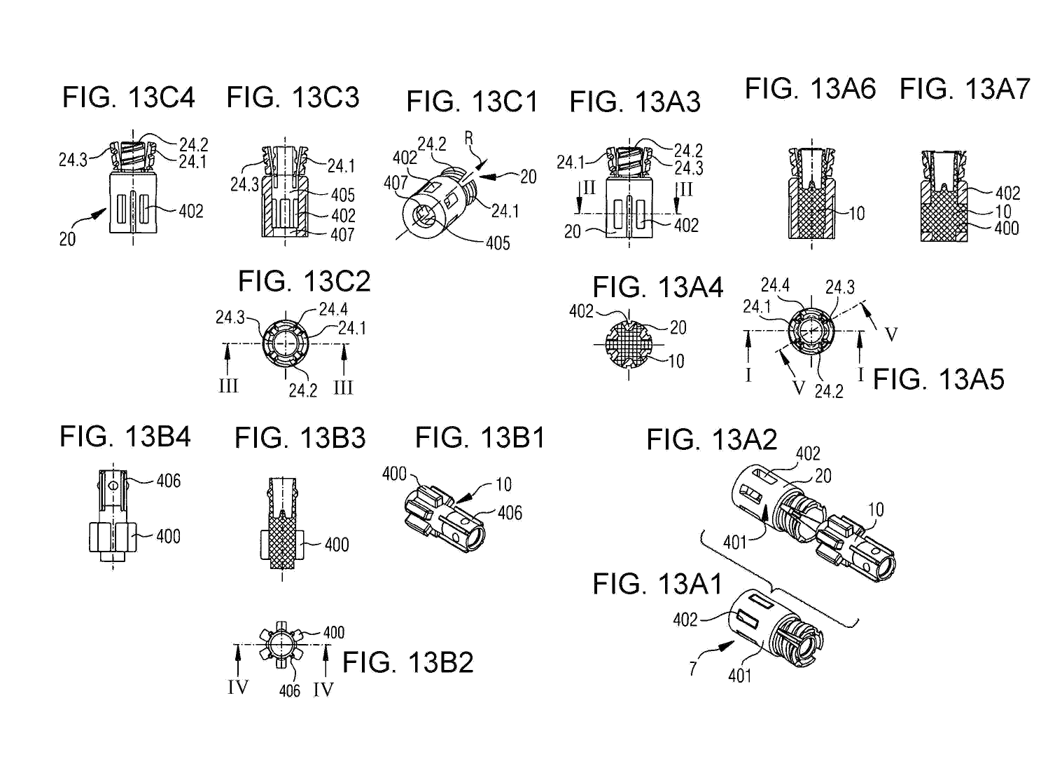

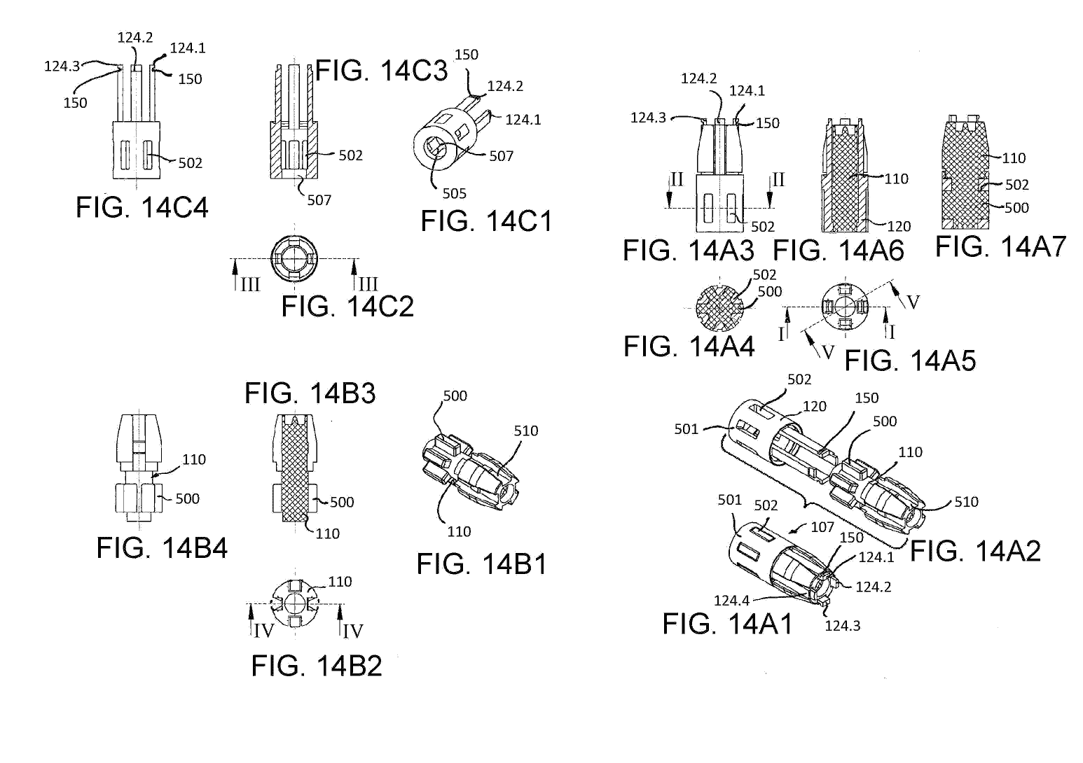

A first embodiment of an alternative closure cap is shown in FIGS. 13A1 to 14C4 which is characterized in that the hard component or respectively the hard region includes openings or grooves into which at least one rib or protrusion 400 of base element 10 consisting of elastomer or the elastomer component engage. The two embodiments according to FIGS. 13A1 to 13C4 differ from the embodiment according to FIGS. 14A1 to 14C4 in that in one case in the embodiment according to FIGS. 13A1 to 13C4 the hard component includes an expansion component with a thread, whereas in the embodiment according to FIGS. 14A1 to 14C4 the hard component includes expansion elements with cams.

In FIGS. 13A1 to 13C4 the entire closure cap is again designated reference number 7. FIG. 13A1 shows the closure cap with the base component or elastic component 10 and the hard part or component 20 in an assembled state. FIG. 13A2 is an exploded view of the closure cap 7 showing the two components, namely hard component 20 and elastic component 10. FIG. 13A3 shows hard component 20 with the elastic component or respectively elastomer component 10 in a side view. Clearly visible are expansion elements 24.1, 24.2, 24.3 and openings 402 in axial section 401 which are distributed over the circumference.

Section II-II through component 20 with elastic component 10 according to FIG. 13A3 is shown in FIG. 13A4. Clearly visible in the sectional view are openings 402 which are distributed over the circumference and which in the assembled state as shown in FIG. 13A1 engage into protrusions 400 of elastic component 10.

FIG. 13A5 is a top view of hard component 20 with the installed elastic component. Expansion elements 24.1, 24.2, 24.3, 24.4 are clearly visible. Sections I-I and V-V according to FIG. 13A5 are respectively shown in FIGS. 13A6 and 13A7. The installation of elastic component 10 is clearly visible. As is clearly shown in section view FIG. 13A7, taken along line V-V, the ribs or respectively protrusions 400 of elastic components 10 engage into openings 402 of hard component 20. Protrusions 400 are clearly visible in FIGS. 13B1 to 13B4 which illustrate elastic component 10 in detail.

In addition to the ribs or respectively protrusions 400 which, as described, engage into openings 402 of hard component 20, thereby preventing an axial and radial displacement of the elastomer component or respectively elastic component 10 inside hard component 20, elastic component 10 includes additional ribs 406 which engage into the spaces of expansion elements 24.1, 24.2, 24.3, 24.4 of the hard component. The section view shown in FIG. 13B3 of the elastic component in FIG. 13B2 taken along line IV-IV, as well as the side view according to FIG. 13B4, of elastic component 10 is identified with the same reference numbers as were designated previously. FIGS. 13C1 to 13C4 are views of hard components 20, whereby FIG. 13C1 is a three-dimensional view of hard component 20 with window cut outs 402. FIG. 13C3 is a section view of the hard component in FIG. 13C2 taken along line III-III. Clearly recognizable is through-opening 405 extending in the axial direction through the hard component, the opening having a circular cross section 407. Even though the cross section of opening 405 is illustrated to be round, this is not mandatory. The opening could also be oval or polygonal. FIG. 14C2 is a top view, FIG. 14C3 is a section view of the hard component 20 in FIG. 14C2 taken along line III-III, and FIG. 14C4 is a side view of hard component 20.

Instead of a thread, one embodiment of hard component 20 could also provide that the expansion component of hard component does not include threading, but only cams 150 as in the embodiment according to FIGS. 7A to 12B. Same components as in FIG. 7A to 12B are identified with same reference numbers, in particular the expansion elements with cams 150 with reference numbers 124.1, 124.2, 124.3 and 124.4, as well as the closure cap with reference number 107.

FIG. 14A1 illustrates again the closure including hard component 120 and elastic component 110 in an assembled or installed state, and FIG. 14A2, an exploded view, illustrates the closure in a disassembled state. The hard component is identified as 120, the soft component or part with 110. Same components as in FIGS. 13A1 to 13C4 are identified with numbers increased by 100.

FIG. 14A3 specifies a side view of the assembled component, and FIG. 14A4 shows a section view of the closure in FIG. 14A3 taken along line II-II. FIG. 14A5 shows a top view of the installed component. FIG. 14A6 is a section view of the closure in FIG. 14A5 taken along line I-I. FIG. 14A7 is a section view of the closure in FIG. 14A5 taken along line V-V. As can be seen in FIG. 14A1, protrusions 500 of elastic component 120 engage with openings 502 of hard component 110 in its installed state and prevent an axial or radial displacement of elastic component 110 with hard component 120. Openings 502 of hard component 120 are arranged in section 501, distributed over the circumference. FIGS. 14B1 to 14B4 show again various views of the elastic component, which is the elastomer component 110. FIG. 14B3 shows a section view of the elastic component in FIG. 14B2 taken along line IV-IV. As can be seen in the three-dimensional view according to FIG. 14B1, component 110 includes, in addition to protrusion 500, grooves 510 into which expansion elements 124.1, 124.2, 124.3, 124.4 of hard component 20 engage, as illustrated in FIGS. 14C1 to 14C4. FIG. 14C1 illustrates the hard component in a three-dimensional view. FIG. 14C3 is a section view of the hard component in FIG. 14C2 taken along line III-III. Clearly visible is through-opening 505 which, in the current example, has a round cross section 507 without being restricted thereto.

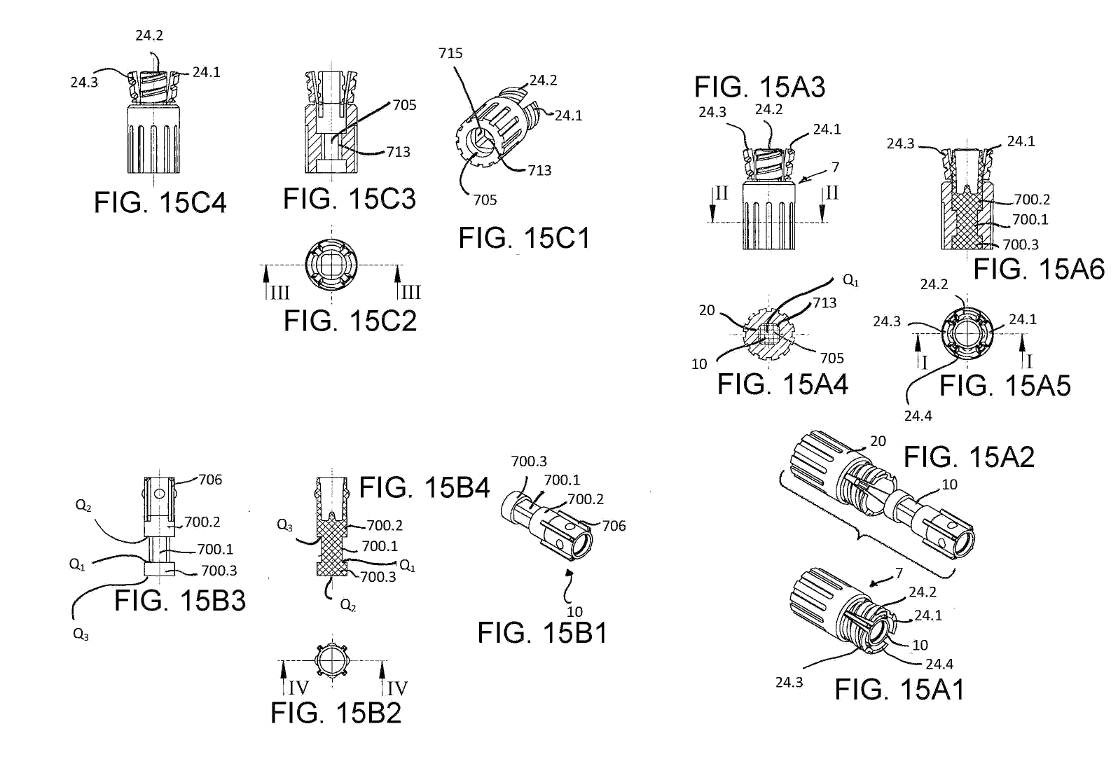

Referring now to FIGS. 15A1 to 18C4, there are shown two embodiments of a closure cap wherein the hard component or respectively the hard region, or respectively hard component 20 has a through-opening 705, including at least one section 713 which has a cross sectional area having an inside contour 715 which is not circular.

FIGS. 15A1 to 15C4 illustrate a first arrangement of such a component wherein the component includes an expansion component having a thread according to FIGS. 1A to 6 which is embedded into expansion elements 24.1, 24.2, 24.3, 24.4 of the hard component. The hard component is again identified with 20, the soft component with 10.

FIG. 15A1 illustrates closure cap 7 in an assembled state, meaning in an installed state, whereby the elastic component is, to a large extent, completely accommodated and at least partially surrounded by the hard part or hard component in through-opening 705. FIG. 15A2, an exploded view of the closure, shows the individual parts or components, namely hard component 20 and elastic component 10.

FIG. 15A3 shows a side view of hard part or hard component 20 with inserted soft component ID. FIG. 15A4 shows a section view taken along line II-II in FIG. 15A3 of elastic component 10. Clearly visible in section II-II is the cross-sectional area of through-opening 705 with an inside contour 713 of the hard component which is not circular. Clearly visible is also soft component 10 which is inserted into through-opening 705 of hard component 20 and which has a first cross-sectional area Q₁ having a non-round outside contour. As can be seen in FIG. 15A4 elastic component 10 is accommodated completely in opening 705 of hard component 20. Moreover, as shown in FIG. 15A6, a section view of the closure according to FIG. 15A5 taken along line I-I, elastic component 10 features three sections in the axial direction. A first section 700.1, a second section 700.2 and a third section 700.3, whereby the second and third sections are wider than first section 700.1. This can also be seen in detail in FIGS. 15B1 to 15B4 which show elastic component 10, preferably the elastomer component in detail. Due to the configuration of first section 700.1 of soft component 10 having a lesser width than sections 700.2 and 700.3 in the radial direction, an axial displacement of elastic component 10 inside hard component 20 is prevented. Moreover, due to the non-round, in particular polygonal, for example square, cross section Q₁ in first section 700.1 of component 10, twisting of component 10 in hard region 20 of closure cap 7 is prevented during unscrewing. This is especially clearly visible in the three-dimensional view in FIG. 15B1. FIG. 15B2 is a top view of the soft component, FIG. 15B3 a side view and FIG. 15B4 is a sectional view taken along line IV-IV in FIG. 15B2. Clearly recognizable in the three-dimensional depiction in FIG. 15B1 are ribs 706 which engage between individual expansion elements 24.1, 24.2, 24.3, 24.4 of the hard component. The hard component is shown in FIGS. 15C1 to 15C4 whereby FIG. 15C1 is to be noted especially which shows the hard component in a three-dimensional view, and the shape of the hard component which corresponds to the soft component and which is characterized by a polygonal cross section 713 in the region of through-opening 705. FIG. 15C3 is a section view of the hard component in FIG. 15C2 along line III-III.

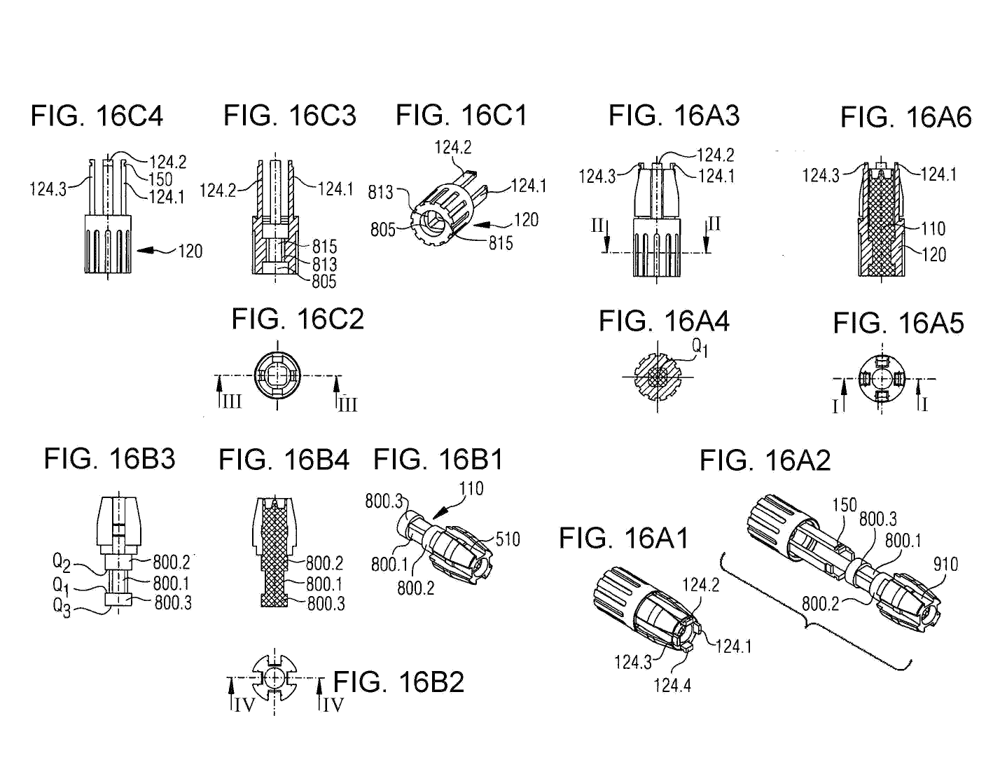

FIGS. 16A1 to 16C4 show an arrangement of a closure cap 107 as described in FIGS. 15A1-15C4 wherein expansion elements 124.1, 124.2, 124.3, 124.4 of hard component 120 do not include a thread but only cams 150, as illustrated in FIGS. 7A to 12B. FIG. 16A2 is an exploded view of the closure according to FIG. 16A1. FIG. 16A4 is a section view of the closure in FIG. 16A3 taken along line II-II. FIG. 16A6 is a section view of the closure in FIG. 16A5 taken along line I-I. Same components are again identified with the same reference numbers. Into the arrangement of the hard component featuring cams 150 in the region of the expansion elements, an elastic element 110 is again introduced which is substantially the same as elastic element 110 in FIGS. 15A1 to 15C4, at least in the region of through-opening 805, which is why same components are identified with reference numbers increased by 100. FIG. 16B4 is a section view of the elastic component according to FIG. 16B2 taken along line IV-IV. As in FIGS. 14A1 to 14C4 the elastic component includes grooves 510 to accommodate the expansion elements. FIG. 16C3 is a section view of the hard component according to FIG. 16C2 taken along line III-III.

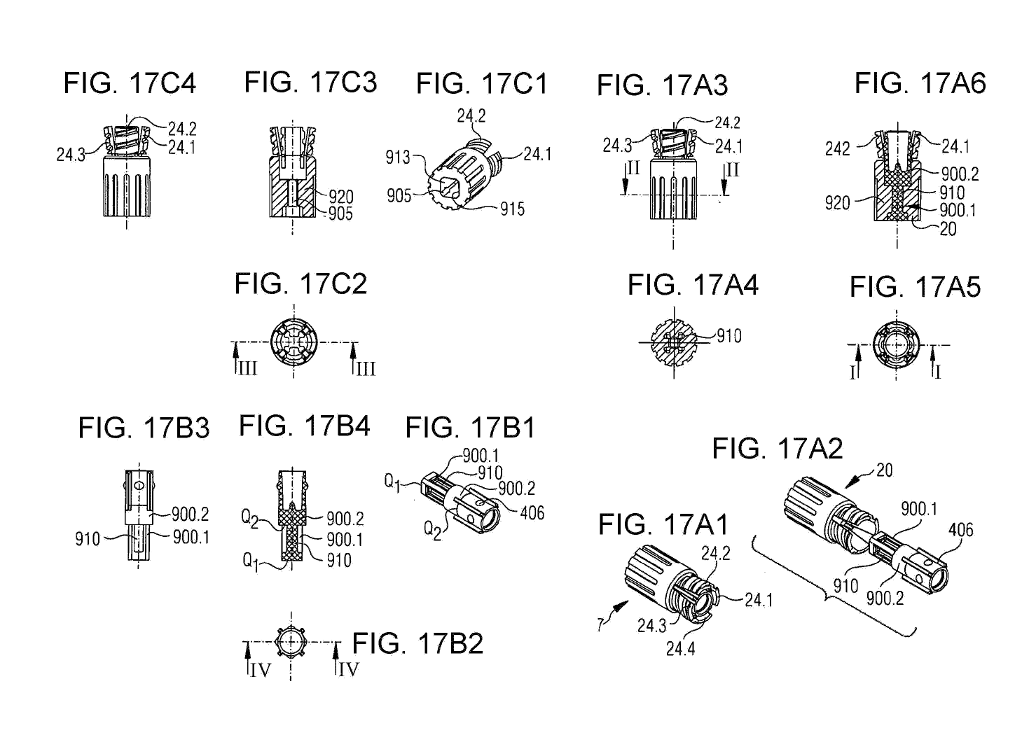

Referring now to FIGS. 17A1 to 18C4, there is shown an additional variation of the embodiment as illustrated in FIGS. 15A1 to 16C4, wherein hard component 20 again features a through-opening 905 with at least one axial section 915 having a cross sectional area with an inside contour which is non-circular, for example oval or polygonal. As in prior figures, the same components are again identified with reference numbers increased by 100. FIG. 17A1 shows the component in assembled state and exploded view FIG. 17A2 shows the hard component 20 as well as elastic component 10 prior to assembly. As can be seen in FIG. 17A2, elastic component 10 has two sections, namely, section 900.1 and section 900.2. FIG. 17A4 shows a section view of the closure in FIG. 17A3 taken along line II-II. First section 900.1 also includes recesses 910 which are embedded in substantially polygonal section 800.1. In an assembled or installed state, protrusions 920 of hard component 20, as illustrated in FIG. 17A6 engage with recesses 910 in region 900.1. In the sectional view in FIG. 17A6, taken along line I-I in FIG. 17A5, individual expansion elements 24.1 with threads are also clearly recognizable in addition to the engagement of protrusions 920 with recesses 910 of elastic component 10.

FIGS. 17B1 to 17B3 show again elastic component 10 and in particular recesses 910 in the region of first section 900.1 wherein elastic component 10 has a non-circular cross section Q₁, in this case having a polygonal cross section. FIG. 17B4 is a section view of the elastic component 10 according to FIG. 17B2 taken along line IV-IV. In the current example cross section Q₂ of second section 900.2 is again circular. Also recognizable are ribs 406 in the region of the expansion elements in assembled state of closure cap 7.

The hard component according to FIGS. 17C1 to 17C4 in an exploded view, a top view, as well as in a section and side view, clearly shows protrusions 920 which engage into the recesses of elastic component 10, thereby preventing an axial displacement of the elastic component during unscrewing of closure cap 7. FIG. 17C3 shows the section view of the hard component taken along line III-III in FIG. 17C2. Also clearly recognizable is the cross section of through-opening 905, which in this example is non-circular but polygonal. Even though previously the opening was always described as passing continuously through the hard component, this is not mandatory. The present invention can also be practiced if the hard component has an opening which is not continuous, but rather in the form of a drilled hole. This is not shown in these drawings but is self-evident to the expert.

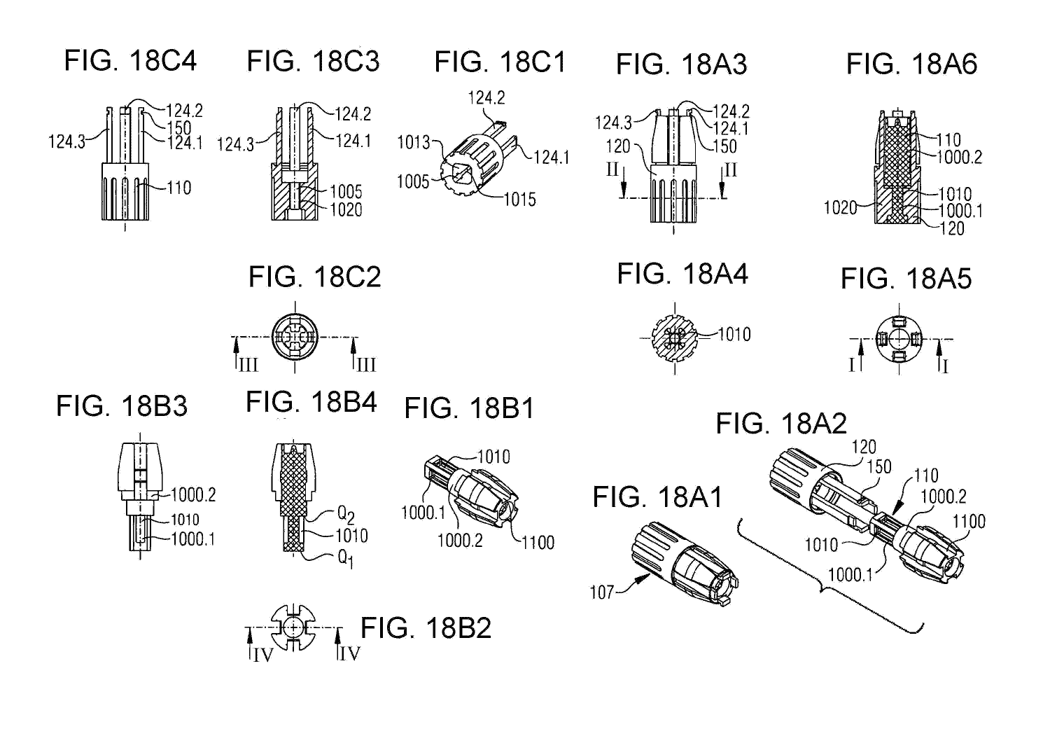

Referring now to FIGS. 18A1 to 18C4, there are shown components wherein the expansion elements of the hard component have cams 150 in the expansion elements instead of the threads. FIG. 18A2 is an exploded view of a closure according to FIG. 18A1. FIG. 18A4 is a section view of the closure in FIG. 18A3 taken along line II-II. FIG. 18A6 is a section view of the closure in FIG. 18A5 taken along line I-I. FIG. 18B4 shows a section view of the elastic component in FIG. 18B2 taken along line IV-IV. FIG. 18C3 is a section view of the hard component in FIG. 18C2 taken along line III-III. Same components are identified as in FIG. 7A to 12B. The arrangement according to FIGS. 18A1 to 18C4 moreover includes an elastic component 110 which is essentially of the same configuration as the elastomer or respectively elastic component 10 in FIGS. 17A1 to 17C4 with all of the therein discussed advantages. Same components as in FIGS. 17A1 to 17C4 are identified with reference numbers increased by 100. It is again recognizable how grooves 1100 in elastic component 10 engage with expansion elements 124.1, 124.2, 124.3, 124.4.

The present invention specifies for the first time a very simple syringe closure system for a sealed closure of a distal opening of a syringe body which guarantees prevention of multiple uses, in particular repeated screwing on of the closure cap. The present invention moreover is formed of very few components, thus ensuring simple production. Due to the selection of the elastomers for the base element, or respectively base component, and hard components for the hard regions which are equipped with the outside thread, it is possible to produce a closure cap which can be realized with all conventional sterilization methods and allows widespread application, in addition to glass syringes, also with syringes consisting of thermoplastics.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A closure for closing a distal opening of a syringe body in a sealing manner, the closure comprising:
a fastening element arrangeable on the syringe body around the distal opening and having an inside thread; and
a closure cap detachably connected with said fastening element and having an outside thread on at least one part of said closure cap, said outside thread configured for engaging said inside thread of said fastening element defining an initial state, said at least one part of said closure cap being under tension in said initial state such that after unscrewing said closure cap from said inside thread of said fastening element, said at least one part of said closure cap is pressed out over said inside thread of said fastening element such that said closure cap cannot be returned to said initial state once said closure cap is unscrewed, said closure cap being configured for closing said distal opening in a sealing manner.

2. The closure according to claim 1, the closure being a syringe closure configured for closing the distal opening of a syringe cone of the syringe body and said fastening element being arranged around said syringe cone.

3. The closure according to claim 1, the syringe body being formed of at least one of a glass material and a thermoplastic material.

4. The closure according to claim 2, said closure cap including a base element formed of an elastomer and having at least one hard region.

5. The closure according to claim 4, said tension in said initial state being introduced into said hard region of said closure cap.

6. The closure according to claim 5, wherein said hard region provides said outside thread and is mechanically connected with said base element.

7. The closure according to claim 6, said base element being configured such that it surrounds only a short section of the distal opening of the syringe cone such that said base element is held on the distal opening of the syringe cone only with said hard region with said outside thread.

8. The closure according to claim 7, said hard region being formed as an expansion component including at least one expansion element, said expansion component configured to expand after being unscrewed from said closure cap and having a diameter greater than a diameter of said fastening element with said inside thread.

9. The closure according to claim 8, said base part being formed of a thermoplastic material.

10. The closure according to claim 9, said thermoplastic material being an elastomer.

11. The closure according to claim 10, said elastomer being one of a thermoplastic elastomer, butyl rubber, chlorobutyl rubber, bromobutly rubber and poly-isoprene rubber permissible for pharmaceutical applications.

12. The closure according to claim 11, said hard region of said closure cap including at least one axial section having at least one recess and said elastomer base element having an additional axial section including at least one radially projecting protrusion distributed over a circumference of said additional axial section and arranged such that said at least one radially projecting protrusion corresponds with and is accommodated by said at least one recess of said axial section of said hard region of said closure cap.

13. The closure according to claim 12, said at least one recess being at least one of an opening and a plurality of grooves distributed over a circumference of said at least one axial section of said hard region of said closure cap.

14. The closure according to claim 13, said hard region being formed as a bushing having said opening extending in an axial direction to accommodate said base element in the assembled state of said closure cap.

15. The closure according to claim 14, said bushing being an outer cap having a through opening.

16. The closure according to claim 15, wherein the at least one recess in said axial section of said hard region of said closure cap is a substantially longitudinal opening in said bushing.

17. The closure according to claim 16, wherein said at least one radially projecting protrusion extends through a corresponding said at least one recess of said hard region and protrudes over a circumference of said bushing.

18. The closure according to claim 15, said through-opening in said hard region of said closure cap having a cross-section which is one of circular, oval-shaped and polygonal.

19. The closure according to claim 11, wherein said hard region is formed as said bushing having said at least one opening in said axial direction to accommodate said base element in said assembled state of said closure cap, said at least one opening in said at least one axial section having a cross sectional area with an inside contour that is not circular.

20. The closure according to claim 18, wherein said cross sectional area has an inside contour that is one of oval and polygonal.

21. The closure according to claim 20, said base element having at least two axial sections in said axial direction, said at least two axial sections including a first axial section having a first cross sectional area and a second axial section having a second cross sectional area, wherein at least said first cross sectional area having an outside contour which corresponds with said inside contour of said at least one opening of said hard part in said axial section of said hard part.

22. The closure according to claim 21, said first axial section of said base element having at least one recess into which said at least one protrusion of said hard component protruding over said inside contour of said at least one opening of said hard part engages.

23. The closure according to claim 22, said base element including a third axial section having a third cross sectional area.

24. The closure according to claim 23, at least one of said second cross sectional area and said third cross sectional area is larger than said first cross sectional area.

25. The closure according to claim 24, said base element including one of a thermoplastic elastomer, butyl rubber, chlorobutyl rubber, bromobutyl rubber or poly-isoprene rubber which is suitable for pharmaceutical applications.

26. A closure cap for sealing an opening, the closure cap comprising:
at least one hard region including at least one axial section having at least one recess; and
at least one base element formed of one of an elastomer and an elastic component, said at least one base element having an additional axial section including at least one radially projecting protrusion distributed over a circumference of said additional axial section and arranged such that said at least one protrusion of said base element is accommodated by a corresponding said at least one recess of said at least one hard region in an assembled state of the closure cap.

27. The closure cap according to claim 26, said at least one recess in said hard region being at least one of a plurality of grooves distributed over a circumferences of said additional axial section and an at least one opening.

28. The closure cap according to claim 27, wherein said at least one hard region is a bushing having an opening in an axial direction to accommodate said base element in said assembled state of the closure cap.

29. The closure cap according to claim 28, said bushing being an outer cap and said opening being a through-opening in said outer cap.

30. The closure cap according to claim 29, said through-opening in said outer cap being a substantially longitudinal opening.

31. The closure cap according to claim 30, wherein said at least one protrusion of said base element extends through said through-opening in said outer cap and protrudes over a circumference of said outer cap.

32. The closure cap according to claim 30, wherein said through-opening in said outer cap has a cross section which is one of circular, oval and polygonal.

33. A closure cap for sealing an opening, the closure cap including at least one base element formed by an elastomer and at least one hard region, said hard region being a bushing having an opening in an axial direction to accommodate said base element in an assembled state of the closure cap, said opening having a cross sectional area in at least one axial section having an inside contour which is not circular.

34. The closure cap according to claim 33, said bushing being an outer cap and said opening in said outer cap being a through-opening.

35. The closure cap according to claim 33, said inside contour of said opening in said bushing being one of oval and polygonal.

36. The closure cap according to claim 35, said base element having at least two axial sections in said axial direction, said at least two axial sections including a first axial section having a first cross sectional area and a second axial section having a second cross sectional area, wherein at least said first cross sectional area having an outside contour corresponding with said inside contour of said through-opening of said outer cap.

37. The closure cap according to claim 26, said first axial section of said base element having at least one recess into which at least one protrusion of said hard region protruding over said inside contour of said opening of said hard region engages.

38. The closure cap according to claim 36, said base element further comprising a third axial section having a third cross sectional area.

39. The closure cap according to claim 38, wherein at least one of said second cross sectional area and said third cross sectional area is larger than said first cross sectional area.

40. The closure cap according to claim 33, said at least one base element including one of a thermoplastic elastomer, butyl rubber, chlorobutyl rubber, bromobutyl rubber and poly-isoprene rubber for pharmaceutical applications.

41. A syringe closure for closing a distal opening of a syringe cone of a syringe body, said syringe closure comprising:
a fastening element arrangeable on the syringe body around the distal opening of the syringe cone, said fastening element being an expansion component having at least one expansion element; and
a closure cap detachably connected to said fastening element using a material-to-material connection connected in a region of said fastening element, said closure cap configured for closing the distal opening in a sealing manner.

42. The syringe closure according to claim 42, said closure cap in said region of said fastening element being exclusively material-to-material connected with said expansion element.

43. The syringe closure according to claim 41, said material-to-material connection being welded.

44. The syringe closure according to claim 43, said closure cap being at least one of laser welded and ultrasonically welded to said fastening element.

45. The syringe closure according to claim 44, said closure cap being material-to-material connected in a region of an outside thread with said fastening element.
